(19) United States
(12) Patent Application Publication
(10) Pub. No.: US 2004/0162598 A1
(43) Pub. Date: Aug. 19, 2004

(54) Composite flexible and conductive catheter electrode and their method of construction

(76) Inventor: Erick Keenan, Vineland, NJ (US)

Correspondence Address: Erick Keenan, 1322 Forest Grove Road, Vineland, NJ, 08360, US

(21) Appl. No.: 10/367,034

(22) Filed: Feb. 19, 2003

(51) Int. Cl. A61N 1/05

(52) U.S. Cl. 607/116

(57) ABSTRACT

This invention present the idea of a composite flexible thermoplastic (polymer/elastomer) and conductive (thin metal piece) band and also will illustrate an example method from which to construct such bands. One envisioned use for the composite flexible conductive band is that of an electrode band located at the distal tip of a cardiac catheter. The band has advantages for such an application; it has controllable flexibility due to the elastic properties of the flexible (polymer/elastomer) part and continuous uninterrupted electrical current conductance from the one-piece design of the conductive element. The synergy of the components of the composite flexible and conductive bands will help solve problems current electrode bands have and will allow for a freedom in the design of catheter electrode band configurations in the future that current bands now cannot.

### BACKGROUND OF THE INVENTION

1. Field of Invention

This invention relates to the electrophysiological catheter electrode band used in the mapping (measurement of electrical potential), pacing (stimulation of the muscle tissue by pulsing an electrical current) and ablating (burning the tissue by use of high electrical current) of the heart's inner wall. This invention generally relates to catheters and leads used in sensing electrical activity within a patient and administering therapy, and more particular to such catheter and leads incorporating band electrodes configured for flexibility and tractability within the body.

2. Discussion of Related Art

(Rigid metal, conductive adhesive, coils, slotted and thin film electrode bands)

A method of construction of a conductive band electrode are typically now accomplished by using a rigid thin wall, metal tube (example; Pt/Ir alloy, Stainless steel) band which is swaged or adhesive boned in place and over where a conductor wire is tied onto the catheter's flexible polymer (example; PVC, polyurethane) hollow tube. This method of construction has some technical and design drawbacks; some are these are:

1. Rigid metal bands limit catheter flexibility and make the ability of the catheter to reach an area so desired impossible.

2. Rigid metal bands cannot be long in length, more than one centimeter can effect the flexibility and curve radius of the catheter thus preventing the electrode from being placed where required.

3. Rigid metal bands have the possibility of sliding off the catheter and being lost inside the patient.

4. Pressure of the catheter flexible polymer tube and the inside wall of the band typically loosely attach the rigid metal bands to the conductor wire, catheter flexing can than cause intermittence in the electrical signals.

5. Rigid metal band are on the surface of the catheter's polymer tube and has corner edges which is exposed on to the patient's tissue which can cause trauma due to abrasions.

6. Rigid metal bands can also allow fluid leakage under and into the space between the band and the catheter's polymer tube, this can cause loss of signal and has sterility issues.

7. Rigid metal band configuration (number and length of bands) and spacing between bands is also a problem; by trying to place bands less the one millimeter apart difficulties are experienced in manufacturing and performance of the catheter.

Another method of band design construction was conceived as an improvement over rigid metal bands by the use of conductive adhesives bands (adhesives filled with a metal or other conductive powder used as the electrical conductor). This colloidal suspension matrix is flexible and thus allowing for longer bands and better placement of the catheter's electrodes onto the required tissue wall; but it also has some design weaknesses:

1. The randomly dispersed conductor particles depends on a continuos touching (but not joined) to allow for the conductance of electrical current, these abutting connections can easily be broken by the curving and flexing of the catheter's polymer tube which the conductive adhesive is applied over. The resulting problems are intermittent signal and spark gap across the conductive particles.

2. Another potential problem is the loss of conductive particles into the patient's blood stream. If the adhesive bond of a band's conductive particle located at the outer surface is week the electrical energies conducted through the bands matrix could cause the breaking of the polymer adhesive's and conductive particle's bond and therefore liberating the particle into the patient's body.

3. Manufacturing issues such as: the control of accuracy of the band's placement, the uniformity of bands thickness, and control of the chemical science which relates to the polymer's adhesive and cohesive properties between not only the conductive particles and adhesive polymer but, also the adhesive polymer and the catheters polymer tube.

4. Electrical current distribution could also be random and difficult to control because of the powder's distribution pattern throughout the inner wall, middle sections and onto the outer surface of the conductive adhesive bands may varry.

A variation on the rigid metal band electrode is the slotted metal band electrode. The advantage the slotted metal band has is derived from several slots that are placed in a pattern around and throughout a metal band to allow flexibility. Basically this concept is still a rigid metal band and therefore many of the problems which occur in rigid metal bands still occur in the slotted metal bands, or are sometime compounded due to the slots. For example a band's corner edges, which could cause tissue abrasions, in traditional non-slotted rigid metal bands number only two (a leading and a tailing corner edge), this issue has been increased with the slotted metal bands because of its multiple of possible abrading corners. The slots also may harbor foreign matter if not thoroughly cleaned.

Unique from the prior band construction methods is a continuously wound metal wire spring coil which is wrapped around or molded into the outer surface of the catheter's distal tip and connected by a wire to transmit an electrical signal to function as an electrode. This method of band construction also gives flexibility but suffers from the need for special tooling and a higher degree of manufacturing process control. The joints between the coil loops may also harbor foreign matter if not thoroughly cleaned.

The last method compared is called thin film electrodes, which is a thin metal film that is deposit by means of vapor deposition on to the completed assembled catheter's distal tip. This metal band is flexible but the nature of the material is such that over time flexing of the metal will cause stress cracks leading to band to failure. This method has some difficulties in its manufacturing process such as it requires a high technology and the catheter's distal tip must be completely assembled prior to the thin film application

The above concepts have been mentioned so as an attempt to give a clear contrast to the invention of the composite flexible conductive bands and other know ideas.

It also should be noted that the author did not attempted to describe the well-established process of cardiac catheter construction so as not to distract from the focus on the invention and concept of the composite flexible and conductive band

### BRIEF SUMMARY OF THE INVENTION

The general concept of the composite flexible and conductive band is that each of its components uses their individual properties to perform a specific function and then they, with synergy, combine to meet the functional requirements of a 2 to 14 french diameter flexible conductive band electrode. The Flexible (polymer/elastomer) component gives the band; variable flexibility, supports the position of the conductive component, locks the conductive component safely into the band, attaches the band securely to the catheter tube, hermetically seals the conductor wire, can include a radiopaque material and can be colored to allow for visual identification of the band or give pleasing aesthetics. The conductive component (a thin metal piece) are designed to possess an anisotrotic strength, in the desired direction and not in an undesired direction thus allowing flexibility in the required direction. The conductive component's primary functional demand is the ability to carry electrical current continuously with out interruption from the connector wire onto the surface of the electrode band contacting the patent's tissue. While the previous statements described the band components that are the mandatory base parts to accomplish a functional Composite flexible and conductive electrode band; an added benefit of this invention is the ability to incorporated enhancements. Features such as a thin electroplated platinum surface onto the outside of the band might be one example.

The invention of the composite flexible and conductive band succeeds over the following shortcoming and technical difficulties in some other methods of prior art in band design, construction and manufacture for cardiac catheter. Flexibility of the electrode band and the catheter's distal tip can be better than that of the other methods due to this inventions use of delineated functional propose. This specific use allows the utilization of the flexible component 100% for flexibility and the conductor component 100% for electrical requirements with out one component effecting the other ones functionality. Although each component performs their task independently, it is by efficient design that some components are not just neutral in a function but aid in helping the band's performance. An example is how that the conductive component is design to be anisotropic so as not to impair the performance of the flexible component in its functionality. Electrical current is uninterrupted and has no spark gaps, this is ensured by the conductive component's continuous one-piece design that connects the conductor wire and the outer surface of the electrode band without breaks or reliance on just touching. The invention's design has a higher degree of robustness than that of some other electrode concepts. For example, the thin film metal band can flex fatigue and fail where as this invention places no such demands on it. Improved attachment to the catheter tube is achieved over rigid metal bands due to the fact that the bands are recessed and physically and chemically joined as one into the tube to become one unit. The invention has superior resistance to electrically induced polymer bond failure due to the fact the flexible component (the polymer) is not stressed into a dual role use such as the adhesive conductive bands are. The adhesive needs to act both as a tight bonding agent to attach the band onto the tube and hold into itself the conductive powder while it must also perform the task of an elastomer to give the band its flexibility. This invention also allows for precise band placement control down to a space of 0.020″ between conductive bands, conductive band thickness as small as 0.0005″, a method of safety locking the conductive element into the band, and a hermetically seal from fluids intrusion into the catheter inner lumens. Construction methods used to produce this invention are of a low technology in art and can be perform with simple low cost tools by technicians of moderate abilities and training. The simple design of the composite flexible and conductive electrode bands and their attachment onto a catheter lends itself to the potential to have a large degree of automated tooling to produce them. Another advantageous feature of this invention is the use of economical materials in construction of the band electrode, which are readily available from a wide source of suppliers.

### BREIF DESCRIPTION OF THE SEVERAL VIEWS OF THE DRAWING

FIG. 1 Illustration of two band grooves located at a catheter's distal tip; one on the left, which is wired, and one on the right, which is not.

FIG. 2 Illustration of a catheter with two composite flexible and conductive bands attached; one on the right, which is shown sectioned, and one on the left, which is not sectioned.

FIG. 3 Shown are the conductive component on the left and the flexible component on the right.

FIG. 4 Shown is the assembled composite flexible and conductive band in perspective.

FIG. 5 An explode illustration of the arrangement of components for the composite flexible and conductive band.

FIG. 6 Perspective illustration of polymer tube with two composite flexible and conductive bands attached; one on the right, which is shown sectioned, and one on the left, which is not sectioned.

FIG. 7 Illustration of an enlarged sectional view of a composite flexible and conductive band attach to a polymer tube.

### DETAILED DESCRIPTION OF THE INVENTION

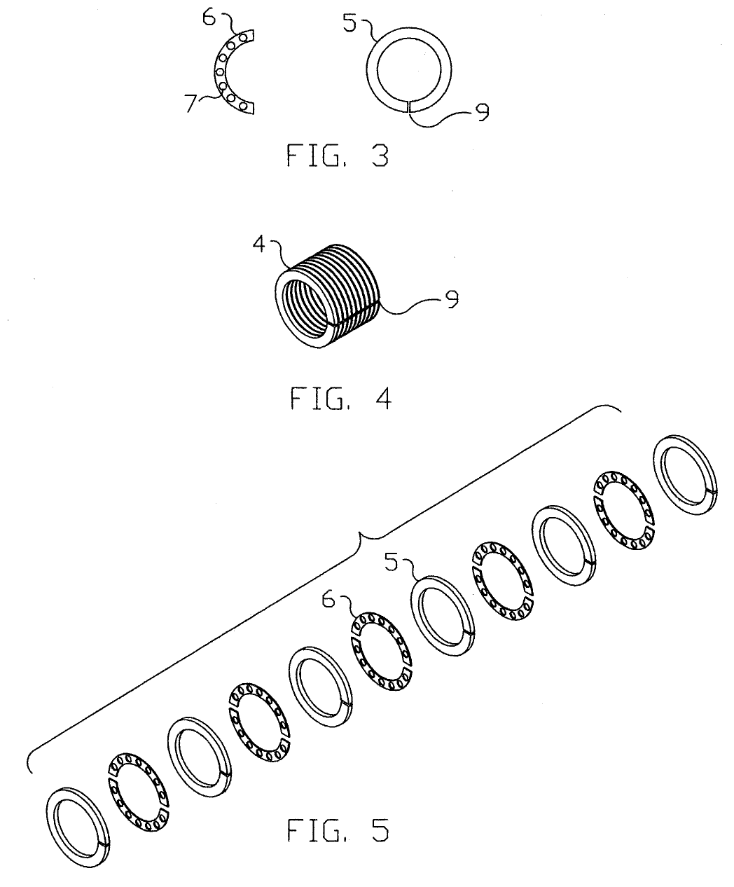

Referring to FIG. 4 is illustrated an assembled composite flexible and conductive band 4 shown in perspective view before its assembly onto a cardiac catheter tube 1 where it performs the function of an electrode. The band 4 is a composite composed of flexible elements 5 and of conductive elements 6, which are shown in FIG. 3. These component elements are arranged in an alternating pattern of flexible 5 and conductive 6 elements in the manor as shown in the exploded perspective illustration in FIG. 5. Uninterrupted electrical currents are carried from the inside hole through the wall to the outside surface of the band 4 by means of the conductive 6 component. The conductive 6 component is made from a thin metal (examples are Gold, Platinum, Silver, Stainless steel, Platinum/Iridium alloy, plated cooper or other suitable metal) sheet between 0.0005″-0.150″ in thickness from which the shape that is shown in FIG. 3 is die stamped, laser cut or by any other effective method produced. The flexible 5 component in made from a thermoplastic polymer/elastomer which may be a polyurethane, PVC or and other suitable thermoplastic polymer/elastomer from which the shape shown in FIG. 3 is die stamped, laser cut, molded or by any other effective method produced. Also the polymer/elastomer used for the flexible 6 component can have compounded into it a radiopaque agent material and/or colorant which are suitable to satisfy the specification of the band's 4 use.

The construction of the band 4 starts with the making of the component parts; the flexible 5 and conductive 6 items are produced in the required number from the materials and by the methods as described above. An alternating pattern of component parts are arranged in a stack up, the first and the last ends are always the flexible 5 parts with as many conductive 6 and flexible 5 pairs required for the length, see FIG. 5 for illustration. The staked assembly is place onto a round rod and loaded into a heated press. A ram presses the stack down and squeezes the assembly until a predetermined dimension is obtained. Under the influence of the pressure of the ram and the temperature that is held around the polymer's material softening point a flowing of polymer through the link holes 7 will occur. The link holes 7 through the conductive 6 component produces an empty area in which each abutting flexible 5 component can melt together making all of the flexible 5 components an unbroken piece of polymer/elastomer. After a short cooling time the press is opened and the band 4 is removed; at this point the composite flexible and conductive band 4 is ready for placement onto a catheter tube as an electrode.

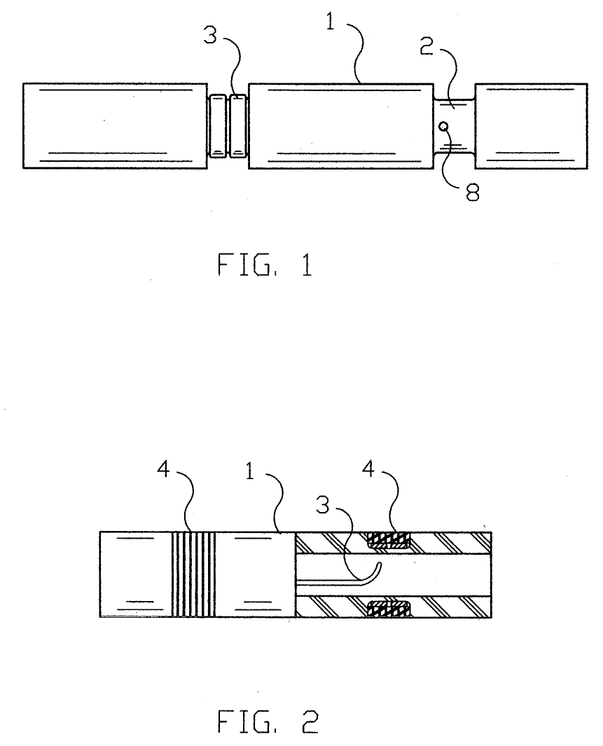

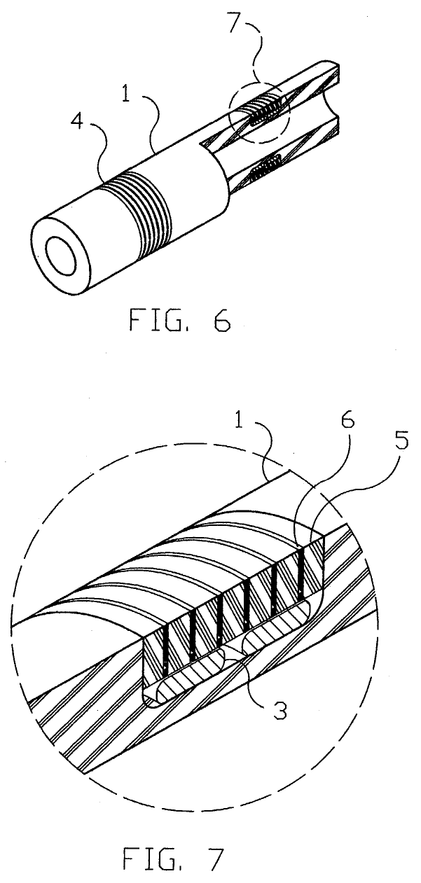

For use as an electrode on a cardiac catheter the first step is to prepare the tube 1 with a wire groove 2 and to wire the groove with a conductor wire 3 which will carry the electrical signal from the band 4 to the proximal end of the catheter. FIG. 1 shows the deformed profile of the wire groove 2 on the right hand side. The profile can be imparted into the tube 1 by either cold plastic deformation or by hot plastic deformation of the polymer tube 1. After the groove 2 has been place where desired a small hole 8 is punched through the wall of the recessed grove 2 area into the tube's center. A standard conductor 3 wire is stripped of its outer insulation and the exposed length is pressed into a flat shape as shown in FIG. 7; the length of stripped and pressed wire relates with the required amount of wire wrap needed. The prepared wire is threaded into the hole 8 with the round end first and puled down to the end of proximal end until the start of the flattened end starts to enter the hole. The wire 3 is then wrapped around the tube 1 as shown in FIG. 1 and the area is now ready for the application of the band 4. The band 4 is opened up from the small slit 9, see FIG. 3 at the six o'clock position and in FIG. 4, like a clamshell and then pushed over the wired area, see FIG. 2 and FIG. 6. After all the required bands 4 are in place the assembled catheter is put into a heated mold which produces closing and sealing at the slit 9 and also bonding together between the tube 1 and the band 4 by melting the polymers together.

1. I claim the invention of a Composite Flexible and Conductive band comprised of the following.
a. The flexible component is made of a 0.001″-0.250″ thick thermoplastic polymer/elastomer such as, for example; polyurethane, PVC, nylon which is made into a round 2-14 French diameter disc with a concentric hole and small slit cut in it.
b. The flexible component can have a radiopaque material and/or colorant mixed into the polymer at various concentrations depending on the application.
c. The conductive component is a 0.0005″-0.1500″ thick metal, such as: Gold, Platinum, Silver, Stainless Steel, Platinum/Iridium alloy, plated cooper, “C” shaped, 2-14 French outer diameter disc with a pattern of safety locking link holes radially spaced through it.

2. I claim the invention of a preferred method of construction of a Composite Flexible and Conductive band, which is performed as follows.
a. Die stamp, mold or by any other effective method, manufacture the flexible component to the prescribed design specifications in the required numbers.
b. Die stamp, laser cut or by any other effective method, manufacture the conductive component to the prescribed design specifications in the required numbers.
c. An arrangement of alternating flexible and conductive components are stacked into a heated press to a length specified which can be from 0.003″ to how ever long depending on the requirements.
d. The ends of the stack are always the flexible component and can be made of a different polymer than the other flexible components.
e. After heating the flexible components to the polymer's softening point and applying the pressure of the press's ram, flowing polymer will fill into the link hole area and join with the abutting flexible components to form a continuous piece; after cooling and removal from the press the band is ready for use.

3. I claim the invention of the flexible and conductive band in its use as a cardiac catheter electrode as described in the following method of construction for that use.
a. Preparation to mount the band electrode onto the catheter's tube must begin with the formation of a wire groove, the punching of a hole in the grooved area, the wire being threaded from the distal to the proximal tube ends and then the wrapping of the flatten section of conductor wire around the groove.
b. Tube groove spacing can be as close as 0.020″ between them.
c. The band is opened at the slit like a clamshell enough to allow the band to slip over and into position at the wire groove.
d. After a final processing of the catheter in a heated press, which closes and seals all open areas between the band and tube by melting the polymers together, the assembly at this point is a functioning Flexible and Conductive catheter electrode.
